(19) United States
(12) Patent Application Publication
(10) Pub. No.: US 2023/0191163 A1
(43) Pub. Date: Jun. 22, 2023

(54) Oral Care Compositions

(71) Applicant: Colgate-Palmolive Company, New York, NY (US)

(72) Inventors: Vivian MUI, Middlesex, NJ (US); Hallena Strotman, Somerset, NJ (US); Leighton Davies-Smith, Lebanon, NJ (US); Jianbong Qiu, Warren, NJ (US); Robert Vogt, Princeton Junction, NJ (US)

(73) Assignee: Colgate-Palmolive Company, New York, NY (US)

(21) Appl. No.: 18/064,628

(22) Filed: Dec. 12, 2022

Related U.S. Application Data
(60) Provisional application No. 63/290,227, filed on Dec. 16, 2021.

(51) Int. Cl. A61Q 11/00; A61K 8/22; A61K 8/81; A61K 8/34

(52) U.S. Cl. CPC A61Q 11/00; A61K 8/22; A61K 8/342; A61K 8/8111; A61K 8/8147

(57) ABSTRACT

Disclosed herein are oral care compositions useful for whitening teeth. The oral care composition may comprise a peroxide source present in an amount to provide from about 0.01 to about 1 wt.% peroxide; a base in an amount up to about 5 wt.%; benzyl alcohol present in an amount from about 0.2 to about 1 wt.%, wherein the oral care composition has a pH of about 8 to about 12 and all weight percentages are based on the total weight of the oral care composition. Methods of making and using the oral care compositions are also provided.

### CROSS-REFERENCE TO RELATED APPLICATION

This application claims the benefit of priority from U.S. Provisional Application No. 63/290,227 entitled “ORAL CARE COMPOSITIONS” and filed Dec. 16, 2021, the contents of which is hereby incorporated herein in its entirety.

### BACKGROUND

Conventional oral care products (e.g., toothpastes, gels, mouth rinse, etc.) often utilize whitening agents to effect teeth whitening. For example, conventional toothpastes containing peroxides (e.g., hydrogen peroxide) are often utilized to oxidize chromophores either bound to surfaces of teeth or accumulated beneath the teeth surface, to thereby whiten the teeth. However, peroxide compounds are highly reactive, and consequently difficult to formulate. Moreover, hydrogen peroxide is unstable and can spontaneously decompose to form oxygen gas (O₂) and water. Decomposition is particularly harmful during storage of the oral care product, where the decomposition lowers the oxidizing power of the composition and the remaining formulation may lack enough remaining peroxide to effectively clean and whiten teeth.

To overcome the decomposition effects, some dentifrice compositions are formulated to initially comprise very high levels of peroxide. The aim of employing very high amounts of peroxide is so that at the time a user applies the dentifrice composition, the dentifrice composition still has a moderate to high level of peroxide, after accounting for degradation of some of the peroxide. However, the exact amount of peroxide delivered on application may depend largely on how long and under what conditions the dentifrice has been stored and, therefore, may still yield variable peroxide levels. More so, certain geographical regions restrict the amount of peroxide which may be included within consumer products.

As such, there is a need for improved peroxide-containing oral care compositions containing a low amount of peroxide where such compositions still exhibit strong whitening efficacy and provide for compositional stability.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

Applicants have discovered that utilization of certain components within an oral care composition having low amounts of peroxide provides for efficacious whitening performance and compositional stability. Furthermore, such compositions may be combined with other active ingredients to deliver oral care health benefits in addition to whitening.

Thus, in one aspect, the invention provides an oral care whitening composition comprising a peroxide source present in an amount to provide from about 0.1% to about 1% of peroxide by weight of the composition; a synthetic polyacrylic acid polymer present in an amount from about 1% to about 5% by weight of the composition; a poloxamer present in an amount from about 0.2% to about 1% by weight of the composition; benzyl alcohol present in an amount from about 0.2% to about 1% by weight of the composition; and an orally acceptable carrier present in an amount from about 80% to about 97% by weight of the composition; wherein the pH of the composition is from about 5 to about 6. In certain embodiments, the peroxide source is selected from hydrogen peroxide, a peroxide whitening complex, and combinations thereof. In certain embodiments, the peroxide source is present in an amount to provide about 0.1% to about 0.5% by weight of the composition. In certain embodiments, the synthetic polyacrylic acid polymer is a carbomer. In certain embodiments, the synthetic polyacrylic acid polymer is present in an amount from about 1% to about 3.5% by weight of the composition. In certain embodiments, the poloxamer is a nonionic surfactant composed of blocks of polyethylene glycol (PEG) and polypropylene glycol (PPG). In certain embodiments, the poloxamer is present in an amount from about 0.3% to 0.8% by weight of the composition. In certain embodiments, the benzyl alcohol is present in an amount from about 0.2 to about 1% by weight of the composition. In certain embodiments, the orally acceptable carrier is selected from glycerin, water, polymers and/or copolymers of polyethylene glycol, of ethylene oxide propylene oxide, and of silicone, and combinations thereof. In certain embodiments, the orally acceptable carrier is present in an amount from about 90% to about 97% by weight of the composition. In certain embodiments, the composition further comprises sodium hydroxide. In certain embodiments, the sodium hydroxide is present in an amount from about 0.3% to about 0.45% by weight of the composition. In certain embodiments, the composition further comprises a sweetener, present in an amount from about 0.05% to about 0.4%, from about 0.08% to about 0.3%, or from about 0.1% to about 0.3%, by weight of the composition. In certain embodiments, the pH of the composition is from about 5.2 to about 5.5. In certain embodiments, the composition further comprises peppermint flavor. In certain embodiments, the peppermint flavor is present in an amount from about 0.05% to about 0.4%, from about 0.08% to about 0.3%, or from about 0.1% to about 0.3%, by weight of the composition.

In other embodiments, the invention is directed towards an oral care product comprising mixing a first amount of a first oral care composition according to any previously described embodiment above with about an equal amount of a second composition comprising a thickening agent; a carbonate; a bicarbonate; and an orally acceptable carrier present in an amount from about 80% to about 97% by weight of the composition; wherein the pH of the second composition is from about 10 to about 11, wherein the mass ratio of carbonate to bicarbonate is from about 12:1 to about 4:1; and wherein the second composition has a viscosity from about 200,000 to about 310,000 cP. In certain embodiments, the pH of the oral care product is from about 9 to about 10. In certain embodiments, the second composition has a viscosity from about 210,000 to about 290,000 cP. In certain embodiments, the mass ratio of carbonate to bicarbonate is from about 10:1 to about 7:1. In certain embodiments, the thickening agent of the second composition is selected from carboxyvinyl polymers, carrageenan, hydroxyethyl cellulose, laponite, water soluble salts of cellulose ethers such as sodium carboxymethylcellulose and sodium carboxymethyl hydroxyethyl cellulose, natural gums such as gum karaya, xanthan gum, gum arabic, and gum tragacanth and combinations thereof. In certain embodiments, the thickening agent of the second composition is present in an amount from about 1% to about 8% by weight of the composition. In certain embodiments, the second composition further comprises a sweetener, present in an amount from about 0.05% to about 1%, from about 0.1% to about 0.8%, or from about 0.4% to about 0.6%, by weight of the composition.

In other embodiments, the invention is directed towards a method of whitening teeth, the method comprising applying to the surface of the teeth an oral care whitening composition comprising a peroxide source present in an amount to provide from about 0.1% to about 1% of peroxide by weight of the composition; a synthetic polyacrylic acid polymer present in an amount from about 1% to about 5% by weight of the composition; a poloxamer present in an amount from about 0.2% to about 1% by weight of the composition; benzyl alcohol present in an amount from about 0.2% to about 1% by weight of the composition; and an orally acceptable carrier present in an amount from about 80% to about 97% by weight of the composition; wherein the pH of the composition is from about 5 to about 6. In certain embodiments, the peroxide source is selected from hydrogen peroxide, a peroxide whitening complex, and combinations thereof. In certain embodiments, the peroxide source is present in an amount to provide about 0.1% to about 0.5% by weight of the composition. In certain embodiments, the synthetic polyacrylic acid polymer is a carbomer. In certain embodiments, the carbomer is present in an amount from about 1% to about 3.5% by weight of the composition. In certain embodiments, the poloxamer is a non-ionic surfactant composed of blocks of polyethylene glycol (PEG) and polypropylene glycol (PPG). In certain embodiments, the poloxamer is present in an amount from about 0.3% to 0.8% by weight of the composition. In certain embodiments, the benzyl alcohol is present in an amount from about 0.2 to about 1% by weight of the composition. In certain embodiments, the orally acceptable carrier is selected from glycerin, water, polymers and/or copolymers of polyethylene glycol, of ethylene oxide propylene oxide, and of silicone, and combinations thereof. In certain embodiments, the orally acceptable carrier is present in an amount from about 90% to about 97% by weight of the composition. In certain embodiments, the composition further comprises sodium hydroxide. In certain embodiments, the sodium hydroxide is present in an amount from about 0.3% to about 0.45% by weight of the composition. In certain embodiments, the composition further comprises a sweetener, present in an amount from about 0.05% to about 0.4%, from about 0.08% to about 0.3%, or from about 0.1% to about 0.3%, by weight of the composition. In certain embodiments, the composition is from about 5.2 to about 5.5. In certain embodiments, the composition further comprises peppermint flavor. In certain embodiments, the peppermint flavor is present in an amount from about 0.05% to about 0.4%, from about 0.08% to about 0.3%, or from about 0.1% to about 0.3%, by weight of the composition. In certain embodiments, the method further comprises mixing the whitening composition with a second composition prior to applying to the surface of the teeth, the second composition comprising a thickening agent; a carbonate; a bicarbonate; and an orally acceptable carrier present in an amount from about 80% to about 97% by weight of the composition; wherein the pH of the second composition is from about 10 to about 11, wherein the mass ratio of carbonate to bicarbonate is from about 12:1 to about 4:1; and wherein the second composition has a viscosity from about 200,000 to about 310,000 cP. In certain embodiments, the whitening composition is mixed with the second composition in a weight ratio of about 1:7 to about 7:1. In certain embodiments, the pH of the mixed composition is from about 9 to about 10. In certain embodiments, the second composition has a viscosity from about 210,000 to about 290,000 cP. In certain embodiments, the mass ratio of carbonate to bicarbonate is from about 10:1 to about 7:1. In certain embodiments, the thickening agent of the second composition is selected from carboxyvinyl polymers, carrageenan, hydroxyethyl cellulose, laponite, water soluble salts of cellulose ethers such as sodium carboxymethylcellulose and sodium carboxymethyl hydroxyethyl cellulose, natural gums such as gum karaya, xanthan gum, gum arabic, and gum tragacanth and combinations thereof. In certain embodiments, the thickening agent of the second composition is present in an amount from about 1% to about 8% by weight of the composition. In certain embodiments, the second composition further comprises a sweetener, present in an amount from about 0.05% to about 1%, from about 0.1% to about 0.8%, or from about 0.4% to about 0.6%, by weight of the composition.

Further areas of applicability of the present disclosure will become apparent from the detailed description provided hereinafter. It should be understood that the detailed description and specific examples, while indicating the preferred embodiment of the disclosure, are intended for purposes of illustration only and are not intended to limit the scope of the disclosure.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other applications and methods. It is to be understood that the invention is not limited in its application to the details of any particular embodiment shown. The terminology used herein is for the purpose of description and not to limit the invention, its application, or uses.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, “containing”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range.

Unless otherwise specified, all percentages and amounts expressed herein and elsewhere in the specification should be understood to refer to percentages by weight of the total composition. Reference to a molecule, or to molecules, being present at a “wt.%” refers to the amount of that molecule, or molecules, present in the composition based on the total weight of the composition.

According to the present application, use of the term “about” in conjunction with a numeral value refers to a value that may be +/- 5% of that numeral. As used herein, the term “substantially free” is intended to mean an amount less than about 5 weight%, less than 3 weight%, 1 wt.%; preferably less than about 0.5 wt.%, and more preferably less than about 0.25 wt.% of the composition.

As used herein, the term “effective amount” refers to an amount that is effective to elicit the desired biological response, including the amount of a composition that, when administered to a subject, is sufficient to achieve an effect toward the desired result. The effective amount may vary depending on the composition, the disease, and its severity and the age, weight, etc., of the subject to be treated. The effective amount can include a range of amounts. As is understood in the art, an effective amount may be in one or more doses, i.e., a single dose or multiple doses may be required to achieve the desired endpoint.

All components and elements positively set forth in this disclosure can be negatively excluded from the claims. In other words, the oral care compositions of the instant disclosure can be free, or essentially free of, all components and elements positively recited throughout the instant disclosure. In some instances, the oral care compositions of the present disclosure may be substantially free of non-incidental amounts of the ingredient(s) or compound(s) described herein. A non-incidental amount of an ingredient or compound is the amount of that ingredient or compound that is added into the oral care composition by itself. For example, an oral care composition may be substantially free of a non-incidental amount of an ingredient or compound, although such ingredient(s) or compound(s) may be present as part of a raw material that is included as a blend of two or more compounds.

All ingredients for use in the compositions described herein should be orally acceptable. As used herein, “orally acceptable” may refer any ingredient that is present in a composition as described in an amount and form which does not render the composition unsafe for use in the oral cavity.

As used herein, an “orally acceptable carrier” refers to a material or combination of materials that are safe for use in the compositions of the present invention, commensurate with a reasonable benefit/risk ratio, with which the peroxide source may be associated while retaining significant efficacy. Preferably, the carrier does not substantially reduce the efficacy of the peroxide source. Selection of specific carrier components is dependent on the desired product form, including dentifrices, rinses, gels, and paints. In various embodiments, the carrier is operable to sufficiently adhere the peroxide source against surfaces within the oral cavity to which the composition is administered, without concomitant use of a dental tray, mouthpiece, tape, or similar appliance.

Unless defined otherwise, all technical and scientific terms used herein have the same meanings as commonly understood by one of ordinary skill in the art to which this invention belongs. All patents, patent applications, publications, and other references cited or referred to herein are incorporated by reference in their entireties for all purposes. In the event of a conflict in a definition in the present disclosure and that of a cited reference, the present disclosure controls.

Aspects of the disclosure are directed to oral care compositions/products that achieve greater whitening efficacy and/or improved stability (e.g., viscosity, pH, peroxide level, flavor, color, appearance, and/or odor), while employing a reduced amount of hydrogen peroxide. The inventors surprisingly discovered that certain oral care compositions having benzyl alcohol and, preferably mint flavor, with a pH of about 8 to about 12 can achieve greater whitening efficacy and improved stability while utilizing lower amounts of peroxide.

In accordance with an aspect of the invention, provided is an oral care composition/product that includes a peroxide source in an amount of from about 0.01 to about 2 wt.%; sodium hydroxide in an amount up to about 0.5 wt.%; benzyl alcohol present in an amount from about 0.2 to about 3 wt.%; a base in an amount up to about 5 wt.%; and optionally, mint flavor in an amount up to about 3 wt.%, wherein the oral care composition has a pH of about 8 to about 12 and all weight percentages are based on the total weight of the oral care composition. The oral care composition/product may comprise a first component (e.g., an oral care whitening composition) and a second component (e.g., a pH adjuster composition). In some embodiments, the oral care composition is a two-phase composition, with the first component is the first phase and the second component is the second phase. The first phase and the second phase may be in contact along a single interphase in some embodiments. In other embodiments, the first component and the second component are physically separated. For example, the first component may be contained in a first chamber while the second component is contained in a second chamber of a different or the same container.

In certain aspects, the present disclosure is directed towards oral care whitening compositions containing a low level (e.g., less than about 2%, less than about 1%, less than about 0.8%, less than about 0.5%,) of peroxide. The oral care whitening compositions provide for beneficial whitening efficacy and have compositional stability. In certain embodiments, the oral care composition comprises a peroxide source present in an amount to provide from about 0.1% to about 2% of hydrogen peroxide by weight of the composition; a synthetic polyacrylic acid polymer present in an amount from about 1% to about 5% by weight of the composition; a poloxamer present in an amount from about 0.2% to about 1% by weight of the composition; benzyl alcohol present in an amount from about 0.2% to about 1% by weight of the composition; and an orally acceptable carrier present in an amount from about 80% to about 97% by weight of the composition; wherein the pH of the composition is from about 5 to about 6. In certain embodiments, the invention is an oral care composition comprising: a peroxide source present in an amount to provide from about 0.1% to about 2% of hydrogen peroxide by weight of the composition; a synthetic polyacrylic acid polymer present in an amount from about 1% to about 5% by weight of the composition; a poloxamer present in an amount from about 0.2% to about 1% by weight of the composition; benzyl alcohol present in an amount from about 0.2% to about 1% by weight of the composition; sodium hydroxide present in an amount from about 0.3% to about 0.45% by weight of the composition; and an orally acceptable carrier present in an amount from about 80% to about 97% by weight of the composition; wherein the pH of the composition is from about 5 to about 6. In certain embodiments, the oral care composition presented above is mixed in about a 1:1 mass ratio with a second composition (also referred to as a buffer gel composition) comprising: a thickening agent; a carbonate; a bicarbonate; and an orally acceptable carrier present in an amount from about 80% to about 97% by weight of the composition; wherein the pH of the second composition is from about 10 to about 11, wherein the mass ratio of carbonate to bicarbonate is from about 12:1 to about 4:1; and wherein the second composition has a viscosity from about 200,000 to about 310,000 cP. The mixed composition provides for an oral care composition having improved stability without compromising of whitening efficacy.

The peroxide source may be selected and/or derived from various compounds. In certain embodiments, the peroxide source is selected from peroxide compounds, peroxide whitening complexes, or a combinations of two or more thereof. In certain embodiments, the peroxide is hydrogen peroxide and is in a solution of water. For example, a 35% H₂O₂ solution may contain 35% hydrogen peroxide and 65% water. In certain embodiments, the peroxide is a peroxide whitening complex which contains a bound hydrogen peroxide. In some embodiments, the peroxide whitening complex is a crosslinked polyvinylpyrrolidone complexed with hydrogen peroxide (PVP-H₂O₂), e.g., Peroxydone™ XL-10 (Ashland Specialty Chemical). The whitening complex may contain 10-30% hydrogen peroxide, based on the weight of the whitening complex, e.g., 15-25%, 15-20%, or about 18%. Example peroxide sources include hydroperoxides, hydrogen peroxide, alkali and alkaline earth metal peroxides, organic peroxide compounds, peroxide acids, pharmaceutically acceptable salts thereof, peroxide complexes, and mixtures of two or more thereof. Alkali and alkaline earth metal peroxides include lithium peroxide, potassium peroxide, sodium peroxide, magnesium peroxide, calcium peroxide, barium peroxide and mixtures thereof. Organic peroxy compounds include urea peroxide, glyceryl hydrogen peroxide, alkyl hydrogen peroxides, dialkyl peroxides, alkyl peroxides, peroxide esters, diacyl peroxide, benzoyl peroxide and monoperoxyphthalate, and mixtures of two or more thereof. The peroxide source may be a peroxide acid and their organic acid peroxide salts, such as alkyl peroxide acids and monoperoxyphthalate and mixtures of two or more thereof. In some instances, the peroxide source is an inorganic peroxide acid, perborate salts of alkali and alkaline earth metals, such as lithium, sodium, potassium, magnesium, calcium and barium and its mixtures. In one embodiment, the peroxide source comprises a peroxide salts, peroxide complex, peroxyphosphate, percarbonate, perborate, peroxyphenate, persulfate, calcium perphosphate, sodium perborate, sodium percarbonate, sodium peroxyphosphate, potassium sulfate, hypochlorite, urea peroxide, hydrogen peroxide polymer complex, hydrogen peroxide-polyvinylpyrrolidine a polymer complex, a metal peroxide, zinc peroxide, calcium peroxide, and a mixture of two or more thereof. Non-limiting examples of a peroxide complex may include a crosslinked polyvinylpyrrolidone complexed with hydrogen peroxide (PVP-H₂O₂).

The peroxide source may be present in the oral care whitening composition in an amount ranging from about 0.01 to about 2 wt.%, based on the total weight of the oral care composition. In some instances, the amount of peroxide source in the oral care whitening composition may be from about 0.01 to about 2 wt.%, about 0.01 to about 1.7 wt.%, about 0.01 to about 1.4 wt.%, about 0.01 to about 1.2 wt.%, about 0.01 to about 1 wt.%, about 0.01 to about 0.8 wt.%, about 0.01 to about 0.6 wt.%, about 0.01 to about 0.4 wt.%, about 0.01 to about 0.3 wt.%, about 0.01 to about 0.2 wt.%, about 0.01 to about 0.1 wt.%; about 0.04 to about 2 wt.%, about 0.04 to about 1.7 wt.%, about 0.04 to about 1.4 wt.%, about 0.04 to about 1.2 wt.%, about 0.04 to about 1 wt.%, about 0.04 to about 0.8 wt.%, about 0.04 to about 0.6 wt.%, about 0.04 to about 0.4 wt.%, about 0.04 to about 0.3 wt.%, about 0.04 to about 0.2 wt.%, about 0.04 to about 0.1 wt.%; about 0.07 to about 2 wt.%, about 0.07 to about 1.7 wt.%, about 0.07 to about 1.4 wt.%, about 0.07 to about 1.2 wt.%, about 0.07 to about 1 wt.%, about 0.07 to about 0.8 wt.%, about 0.07 to about 0.6 wt.%, about 0.07 to about 0.4 wt.%, about 0.07 to about 0.3 wt.%, about 0.07 to about 0.2 wt.%, about 0.07 to about 0.1 wt.%; about 0.1 to about 2 wt.%, about 0.1 to about 1.7 wt.%, about 0.1 to about 1.4 wt.%, about 0.1 to about 1.2 wt.%, about 0.1 to about 1 wt.%, about 0.1 to about 0.8 wt.%, about 0.1 to about 0.6 wt.%, about 0.1 to about 0.4 wt.%, about 0.1 to about 0.3 wt.%, about 0.1 to about 0.2 wt.%; about 0.15 to about 2 wt.%, about 0.15 to about 1.7 wt.%, about 0.15 to about 1.4 wt.%, about 0.15 to about 1.2 wt.%, about 0.15 to about 1 wt.%, about 0.15 to about 0.8 wt.%, about 0.15 to about 0.6 wt.%, about 0.15 to about 0.4 wt.%, about 0.15 to about 0.3 wt.%, about 0.15 to about 0.2 wt.%, about 0.15 to about 0.1 wt.%; about 0.25 to about 2 wt.%, about 0.25 to about 1.7 wt.%, about 0.25 to about 1.4 wt.%, about 0.25 to about 1.2 wt.%, about 0.25 to about 1 wt.%, about 0.25 to about 0.8 wt.%, about 0.25 to about 0.6 wt.%, about 0.25 to about 0.4 wt.%; about 0.5 to about 2 wt.%, about 0.5 to about 1.7 wt.%, about 0.5 to about 1.4 wt.%, about 0.5 to about 1.2 wt.%, about 0.5 to about 1 wt.%, about 0.5 to about 0.8 wt.%; about 1 to about 2 wt.%, about 1 to about 1.7 wt.%, about 1 to about 1.4 wt.%, about 1 to about 1.2 wt.%, based on the total weight of the oral care composition. In some embodiments, the peroxide source is present in an amount to deliver a low amount of peroxide, such as, for example, about 0.1 to about 1.0% of peroxide based on the weight of the composition, e.g., 0.1-0.75%, 0.1-0.5%, 0.15-0.4%, or about 0.2%. In certain embodiments, the peroxide source is present in an amount to provide from about 0.1% to about 0.5% of peroxide by weight of the composition. In certain embodiments, the peroxide whitening complex is present in an amount to provide about 0.15% to about 0.3% of peroxide by weight of the composition.

The oral care whitening composition typically includes a thickener in an amount from about 1 to about 10 wt.% based on the total weight of the oral care composition. For example, the oral care whitening composition may include a thickener in an amount of about 1 to about 10 wt.%, about 1 to about 8 wt.%, about 1 to about 6 wt.%, about 1 to about 5 wt.%, about 1 to about 4 wt.%, about 1 to about 3 wt.%; about 2 to about 10 wt.%, about 2 to about 8 wt.%, about 2 to about 6 wt.%, about 2 to about 5 wt.%, about 2 to about 4 wt.%, about 2 to about 3 wt.%; about 3 to about 10 wt.%, about 3 to about 8 wt.%, about 3 to about 6 wt.%, about 3 to about 5 wt.%, or about 3 to about 4 wt.%, including ranges and subranges thereof, based on the total weight of the oral care composition.

The oral care composition may include a thickener chosen from polyacrylic polymer, poloxamers, polysaccharides, carboxymethyl celluloses (CMC), and combinations of two or more thereof. For instance, the thickener may comprise a polymer selected from polyacrylic polymer, polyvinyl pyrrolidone, a polyacrylate, a polymethacrylate, a polyitaconate, an acrylamide, 2-acrylamido-2-methylpropane sulfonic acid (AMPS) and a combination of two or more thereof. Additionally or alternatively, the thickener may comprise one or more of a polymer, fumed silica, sodium stearate, stearic acid, a fatty amphiphile (e.g., stearyl alcohol or cetearyl alcohol), and a combination of two or more thereof. In some embodiments, the polyacrylic polymer is a carbomer. Carbomers are synthetic high-molecular-weight polyacrylic acids cross-linked with allyl sucrose or allyl pentaerythritol and contain between 56 and 68% w/w carboxylic acid groups. Non-limiting examples of carbomers can include, for example, carbomer 934, carbomer 934P, carbomer 940, carbomer 94, carbomer 1342, carbomer copolymers, carbomer homopolymers, carbomer interpolymers, and combinations thereof. Some carbomers are available commercially from B. F. Goodrich as the Carbopol® series. Particularly preferred Carbopols include Carbopol® 934, 940, 941, 956, 974P, ETD2020 and mixtures thereof. In some embodiments, the synthetic polyacrylic acid polymer is selected from Carbopol® 956, Carbopol® ETD2020 and a combination thereof. In some embodiments, the synthetic polyacrylic acid polymer is Carbopol® 956. In some embodiments, the synthetic polyacrylic acid polymer is Carbopol® ETD2020. In some embodiments, the synthetic polyacrylic acid polymer is a combination of Carbopol® 956 and Carbopol® ETD2020. In some embodiments, the synthetic polyacrylic acid polymer is present in an amount of from about 1% to about 5%, from about 1% to about 3.5%, or from about 1.5% to about 2.5%, by weight of the composition.

The oral care composition may comprise a poloxamer. Poloxamers include blocks of polyethylene glycol (PEG) and polypropylene glycol (PPG), for example poly(oxyethylene)-poly(oxypropylene) block copolymers. Poloxamers may have varying contents of ethylene oxide and propylene oxide, leading to a range of chemical structures and molecular weights. In some embodiments, the poloxamer is a nonionic tri-block copolymer comprised of a central hydrophobic chain of polyoxypropylene (poly(propylene oxide)) flanked by two hydrophilic chains of polyoxyethylene (poly(ethylene oxide)). In some implementations, the poloxamer has a high molecular weight. For example, the poloxamer may have a molecular weight of 5,000 Daltons or more, 6,000 Daltons or more, 7,000 Daltons or more, or 8,000 Daltons or more. In one embodiment, the poloxamer has a molecular weight from about 8,500 Daltons to about 12,500 Daltons. In one embodiment, the poloxamer is poloxamer 407, which is commercially available, for example, under the trade name PLURONIC™ F127 from BASF Corporation. In other embodiments, the poloxamer is selected from Poloxamer 338, Poloxamer 407, Poloxamer, 237, Poloxamer, 217, Poloxamer 124, Poloxamer 184, Poloxamer 185, and a combination of two or more thereof. In some embodiments, the poloxamer is present in an amount from about 0.2% to about 1% by weight of the composition. In some embodiments, the poloxamer is present in an amount from about 0.3% to about 0.8% by weight of the composition.

The oral care composition comprises benzyl alcohol. Benzyl alcohol may be present at various concentrations or amounts. In certain embodiments, benzyl alcohol is present in an amount from about 0.2% to about 3% by weight of the composition. In certain embodiments, benzyl alcohol is present in an amount from about 0.2% to about 1%, from about 0.2% to about 0.8%, from about 0.2% to about 0.7%, or from about 0.4% to about 0.6%, by weight of the composition. In certain embodiments, benzyl alcohol is present in an amount from about 0.3% to 0.8% by weight of the composition.

The orally acceptable carrier may comprise water, polymers and/or copolymers of polyethylene glycol, of ethylene oxide propylene oxide, and of silicone. If such copolymers/polymers are used, they may be selected from the commercially available materials PLURAFLO® L4370 and PLURAFLO® L1220 (available from BASF, Wyandotte, Mich., United States of America). In one embodiment such polymer and/or copolymer is an ethylene oxide, propylene oxide block co-polymer of formula (ethylene oxide)x-(propylene oxide)y wherein x is an integer of 80-150, e.g., 100-130, e.g., about 118, and y is an integer 30-80, e.g., about 60-70, e.g., about 66, having an average molecular weight of greater than 5000, e.g., 8000-13000 Da, e.g., about 9800. Block copolymers of ethylene oxide/propylene oxide are useful, but higher molecular weight, e.g., >5000 Da are preferred, e.g., including PLURACARE® L1220 (available from BASE, Wyandotte, Mich., United States of America). Low or medium molecular weight polyethylene glycol, e.g., PEG 400, PEG 600, PEG 800, PEG 1000 and mixtures thereof are also useful. In some embodiments, the orally acceptable carrier is selected from water, glycerin, polymers and/or copolymers of polyethylene glycol, of ethylene oxide propylene oxide, and of silicone, and combinations thereof. In certain embodiments, the orally acceptable carrier is selected from water, polyols, or combinations thereof. In certain embodiments, the orally acceptable carrier is present in an amount from about 80% to about 98% by weight of the composition. In certain embodiments, the orally acceptable carrier is present in an amount from about 80% to about 97%, from about 85% to about 97%, from about 90% to about 97%, or from about 92% to about 97%, by weight of the composition. In certain embodiments, the orally acceptable carrier is present in an amount from about 90% to 97% by weight of the composition.

In some embodiments, the orally acceptable carrier comprises a polyol in an amount from about 1 to about 60 wt.%, based on the total weight of the oral care composition. For instance, the polyol may be present in an amount of about 1 to about 60 wt.%, about 5 to about 60 wt.%, about 10 to about 60 wt.%, about 15 to about 60 wt.%, about 20 to about 60 wt.%, about 25 to about 60 wt.%, about 30 to about 60 wt.%, about 35 to about 60 wt.%; about 1 to about 50 wt.%, about 5 to about 50 wt.%, about 10 to about 50 wt.%, about 15 to about 60 wt.%, about 20 to about 50 wt.%, about 25 to about 50 wt.%, about 30 to about 60 wt.%, about 35 to about 50 wt.%; about 1 to about 40 wt.%, about 5 to about 40 wt.%, about 10 to about 40 wt.%, about 15 to about 40 wt.%, about 20 to about 40 wt.%, about 25 to about 40 wt.%, about 30 to about 40 wt.%, about 35 to about 40 wt.%; about 1 to about 30 wt.%, about 5 to about 30 wt.%, about 10 to about 30 wt.%, about 15 to about 30 wt.%, about 20 to about 60 wt.%, or about 25 to about 30 wt.%, based on the total weight of the oral care composition. The polyol may be chosen from those having from 2 to 15 carbon atoms and at least two hydroxyl groups. For example, the polyol may comprise one or more glycol(s), such as ethylene glycol, butylene glycol, hexylene glycol, pentylene glycol, 1,3-propanediol, diethylene glycol, dipropylene glycol, caprylyl glycol, and a mixture of two or more thereof. Preferably, the polyol comprises glycerin.

The oral care compositions may include a mint flavor. The mint flavor may be present in an amount from about 0.01 to about 5 wt.%, based on the total weight of the oral care composition. For instance, the mint flavor may be present in an amount from about 0.01 to about 5 wt.%, about 0.01 to about 4 wt.%, about 0.01 to about 3 wt.%, about 0.01 to about 2 wt.%, about 0.01 to about 1 wt.%, about 0.01 to about 0.5 wt.%; about 0.05 to about 5 wt.%, about 0.05 to about 4 wt.%, about 0.05 to about 3 wt.%, about 0.05 to about 2 wt.%, about 0.05 to about 1 wt.%, about 0.05 to about 0.5 wt.%; about 0.1 to about 5 wt.%, about 0.1 to about 4 wt.%, about 0.1 to about 3 wt.%, about 0.1 to about 2 wt.%, about 0.1 to about 1 wt.%, about 0.1 to about 0.5 wt.%; about 0.5 to about 5 wt.%, about 0.5 to about 4 wt.%, about 0.5 to about 3 wt.%, about 0.5 to about 2 wt.%, about 0.5 to about 1 wt.%; about 1 to about 5 wt.%, about 1 to about 4 wt.%, about 1 to about 3 wt.%, or about 1 to about 2 wt.%, based on the total weight of the oral care composition. The mint flavor may comprise a peppermint flavor, spearmint flavor, wintergreen flavor, or the like. In some cases, the mint flavor comprises an essential oil, such as peppermint oil, spearmint oil, and/or wintergreen oil.

The oral care composition generally comprises a base. The base may be used to control the pH of the composition. In certain embodiments, the base is a strong base. In certain embodiments, the base is sodium hydroxide, potassium hydroxide, calcium hydroxide, sodium bicarbonate, sodium carbonate, and mixtures thereof. In certain embodiments, the mass ratio of carbonate to bicarbonate is from about 10:1 to about 5:1, from about 10:1 to about 7:1, from about 9.5:1 to about 7.5:1, or from about 8:1 to about 9:1. In certain embodiments, the carbonate is sodium carbonate. In certain embodiments, the bicarbonate is sodium bicarbonate. The base may be present at various concentrations or amounts. For example, a base may be present in the oral care composition in an amount of about 0.1 to about 5 wt.%, about 0.1 to about 4 wt.%, about 0.1 to about 3 wt.%, about 0.1 to about 2 wt.%, about 0.1 to about 1 wt.%; about 0.5 to about 5 wt.%, about 0.5 to about 4 wt.%, about 0.5 to about 3 wt.%, about 0.5 to about 2 wt.%, about 0.5 to about 1 wt.%; about 1 to about 5 wt.%, about 1 to about 4 wt.%, about 1 to about 3 wt.%, about 1 to about 2 wt.%; about 2 to about 5 wt.%, about 2 to about 4 wt.%, or about 2 to about 3 wt.%, including ranges and subranges thereof, based on the total weight of the oral care composition.

In certain embodiments, the base is present in an amount from about 0.25 wt.% to about 0.48 wt.% by weight of the composition. In certain embodiments, the base is present in an amount from about 0.25 wt.% to about 0.45 wt.%, from about 0.25 wt.% to about 0.4 wt.%, from about 0.25 wt.% to about 0.6 wt.%, or from about 0.3 wt.% to about 0.45 wt.%, by weight of the composition. In certain embodiments, the base is present in an amount from about 0.4 wt.% to 0.6 wt.% by weight of the composition.

The oral care composition may have a pH that can vary, but is typically in the range of from about 8 to about 12. For example, the oral care composition may have a pH of about 8 to about 12, about 8 to about 11, about 8 to about 10, about 8 to about 9.5; about 8.5 to about 12, about 8.5 to about 11, about 8.5 to about 10, about 8.5 to about 9.5; about 9 to about 12, about 9 to about 11, or about 9 to about 10, including ranges and subranges thereof.

The viscosity of the oral care whitening composition may vary. However, having a viscosity too low or too high may negatively affect the efficacy and/or stability of the composition. As used herein, the term “viscosity” may refer to the internal resistance to flow exhibited by a fluid (e.g., water) or the ratio of shearing stress to rate of shear, and may be measured in poise or centipoise (cP). The viscosity of the various compositions discussed and described herein may be determined at a temperature of about 25° C. using, e.g., a Brooksfield viscometer and a number 3 spindle. In at least one implementation, the viscosity or target viscosity of the oral care whitening composition may be greater than or equal to about 200,000 cP and less than or equal to about 320,000 cP. For example, the viscosity of the oral care whitening composition may be about 200,000 cP, about 210,000 cP, about 220,000 cP, about 250,000 cP, about 260,000 cP, about 270,000 cP, about 290,000 cP, about 300,000 cP, about 310,000 cP, or about 200,000 cP to about 320,000 cP, about 300,000 cP, about 280,000 cP, about 270,000 cP, about 260,000 cP, or about 250,000 cP. In a typical implementation, the viscosity of the oral care whitening composition is from about 215,000 cP to about 310,000 cP. In certain embodiments, the viscosity of the composition is from about 200,00 ,0 cP to about 320,000 cP. In certain embodiments, the viscosity of the composition is from about 200,00 ,0 cP to about 320,000 cP, from about 210,00 ,0 cP to about 280,000 cP, or from about 215,00 ,0 cP to about 310,000 cP.

The oral care compositions may include one or more additional ingredients including, e.g., non-hydrogen peroxide whitening agents, nonionic surfactants, amphoteric surfactants, cationic surfactants, stannous salts and/or ions thereof, thickening agents, preservatives, emulsify, colorants, pigments, flavoring agents, sweeteners, abrasives, or the like.

The oral care composition of the present invention may include one or more flavoring agents other than mint flavor. Suitable flavoring agents include, but are not limited to, essential oils and various flavoring aldehydes, esters, alcohols, and similar materials. Examples of essential oils includes oils of sassafras, clove, sage, eucalyptus, marjoram, cinnamon, lemon, lime, grapefruit, and orange. Also useful are such chemicals as menthol, carvone, and anethole. In certain embodiments, the oral care composition comprises saccharin, or a combination thereof. The one or more flavoring agents is typically incorporated in the oral composition at a concentration of 0.01 to 3% by weight. In certain embodiments, the composition comprises peppermint present in an amount from about 0.1% to about 1%, from about 0.1% to about 0.75%, or from about 0.1% to about 0.5%, by weight of the composition and saccharin present in an amount from about 0.1% to about 1%, from about 0.1% to about 0.75%, or from about 0.1% to about 0.5%, by weight of the composition.

The oral care composition of the present invention may include one or more sweeteners safe for oral application. The sweetener may be, for example, saccharin, for example sodium saccharin, aspartame, acesulfame, neotame, cyclamate or sucralose; natural high-intensity sweeteners such as thaumatin, stevioside or glycyrrhizin; or such as sorbitol, xylitol, maltitol or mannitol. In certain embodiments, the sweetener is selected from sucralose, saccharin, aspartame, acesulfame, or a combination thereof. The one or more sweeteners may be present in an amount from about 0.01% to about 1% by weight, from about 0.01% to about 0.8%, from about 0.1% to about 0.75%, or from about 0.1 to about 0.5% by weight of the composition. In a certain embodiment, the composition comprises a triple sweetener system of sodium Saccharin, Sucralose and rebaudioside M (Reb M). In certain embodiments, the sweetener comprises saccharin, and is present in an amount from about 0.05% to about 0.4%, from about 0.08% to about 0.3%, or from about 0.1% to about 0.3%, by weight of the composition.

In certain embodiments, the oral care composition of the present invention may be free of, or substantially free of, abrasive. As used herein, the terms “free of abrasive” or “substantially free of abrasive” may refer to a composition that contains abrasive in an amount of less than 5 weight%, less than 3 weight%, less than 1 weight%, less than 01 weight%, less than 0.05 weight%, less than 0.01 weight%, less than 0.005 weight%, or less than 0.0001 weight%, based on a total weight of the oral care composition. In some embodiments, the oral care composition does not contain abrasive. In other embodiments, however, the oral care composition may have from about 1 to about 40 wt.% of an abrasive. For example, the oral care composition may include an abrasive in amount from about 5 to about 35 wt.%, about 10 to about 30 wt.%, about 15 to about 30 wt.%, or about 20 to about 30 wt.%, including ranges and subranges thereof, based on the total weight of the oral care composition. The abrasive may be selected from silica, alumina, insoluble phosphates, calcium carbonate, resinous abrasives, and a combination of two or more thereof.

In certain embodiments, the composition is a gel. For example, the peroxide source, the synthetic polyacrylic acid polymer, poloxamer, benzyl alcohol and other ingredients of the composition may be maintained together with one another within a single phase.

The oral care composition of the present invention may comprise fluoride, such as one or more fluoride ion sources (e.g., soluble fluoride salts). A wide variety of fluoride ion-yielding materials may be employed as sources of soluble fluoride. Illustrative fluoride ion sources include, but are not limited to, sodium fluoride, stannous fluoride, potassium fluoride, sodium monofluorophosphate, fluorosilicate salts, such as sodium fluorosilicate and ammonium fluorosilicate, amine fluoride, ammonium fluoride, and combinations thereof. In some embodiment, the fluoride ion source is sodium monofluorophosphate or sodium fluoride. The amount of the fluoride ion source present in the oral care composition may be greater than 0 weight % and less than 0.8 wt.%, less than 0.7 wt.%, less than 0.6 wt.%, less than 0.5 wt.%, or less than 0.4 wt.%. The fluoride ion sources may be present in an amount sufficient to supply 25 ppm to 5,000 ppm of fluoride ions, generally at least 500 ppm, e.g., 500 to 2000 ppm, e.g., 1000 ppm to 1600 ppm, e.g., 1450 ppm. In certain embodiments, the fluoride ion source is sodium monofluorophosphate and is present in an amount from about 0.01 to about 1.14%, by weight of the composition, including all values in between.

The oral care composition of the present invention may comprise anticalculus agents. Illustrative anticalculus agents may include, but are not limited to, phosphates and polyphosphates (e.g., pyrophosphates), polyaminopropanesulfonic acid (AMPS), hexametaphosphate salts, zinc citrate trihydrate, polypeptides, polyolefin sulfonates, polyolefin phosphates, diphosphonates, and phytate acid or its alkaline salt. In some embodiments, the anticalculus agent comprises tetrasodium pyrophosphate (TSPP), sodium tripolyphosphate (STPP), or a combination thereof.

The oral care composition of the present invention may comprise a basic amino acid in free or salt form. The basic amino acids which can be used in the compositions include not only naturally occurring basic amino acids, such as arginine, lysine, and histidine, but also any basic amino acids having a carboxyl group and an amino group in the molecule, which are water-soluble and provide an aqueous solution with a pH of about 7 or greater. Accordingly, basic amino acids include, but are not limited to, arginine, lysine, citrullene, ornithine, creatine, histidine, diaminobutanoic acid, diaminopropionic acid, salts thereof or combinations thereof. In a particular embodiment, the basic amino acids are selected from arginine, lysine, citrullene, and ornithine. The basic amino acids of the oral care composition may generally be present in the L-form or L-configuration. The basic amino acids may be provided as a salt of a di- or tri-peptide including the amino acid. In some embodiments, at least a portion of the basic amino acid present in the oral care composition is in the salt form. In some embodiments, the basic amino acid is arginine, for example, L-arginine, or a salt thereof. Arginine may be provided as free arginine or a salt thereof. For example, Arginine may be provided as arginine phosphate, arginine hydrochloride, arginine sulfate, arginine bicarbonate, or the like, and mixtures or combinations thereof. The basic amino acid may be provided as a solution or a solid. For example, the basic amino acid may be provided as an aqueous solution. In some embodiment, the amino acid includes or is provided by an arginine bicarbonate solution. For example, the amino acid may be provided by an about 40 wt.% solution of the basic amino acid, such as arginine bicarbonate or alternatively called as arginine carbamate. In some embodiments, the basic amino acid is present in an amount of from 1% to 15%, e.g., from 1% to 10%, from 1% to 5%, from 1% to 3%, from 1% to 2%, from 1.2% to 1.8%, from 1.4% to 1.6%, or about 1.5% by weight of the composition, being calculated as free base form.

The oral care composition of the present invention may comprise a zinc ion source. The zinc ion source may be or include a zinc ion and/or one or more zinc salts. For example, the zinc salts may at least partially dissociate in an aqueous solution to produce zinc ions. Illustrative zinc salts may include, but are not limited to, zinc lactate, zinc oxide, zinc chloride, zinc phosphate, zinc citrate, zinc acetate, zinc borate, zinc butyrate, zinc carbonate, zinc formate, zinc gluconate, zinc glycerate, zinc glycolate, zinc picolinate, zinc proprionate, zinc salicylate, zinc silicate, zinc stearate, zinc tartrate, zinc undecylenate, and mixtures thereof. In some embodiments, the zinc ion source is present in an amount of from 0.01% to 5%, e.g., 0.1% to 4%, or 1% to 3%, by weight of the composition.

The oral care composition of the present invention may include a stannous ion source. The stannous ion source can be a soluble or an insoluble compound of stannous with inorganic or organic counter ions. Examples include the fluoride, chloride, acetate, hexafluorozirconate, sulfate, tartrate, gluconate, citrate, malate, glycinate, pyrophosphate, metaphosphate, oxalate, phosphate, carbonate salts and oxides of stannous. In some embodiments, the stannous ion source is selected from the group consisting of stannous chloride, stannous fluoride, stannous pyrophosphate, stannous formate, stannous acetate, stannous gluconate, stannous lactate, stannous tartrate, stannous oxalate, stannous malonate, stannous citrate, stannous ethylene glycoxide, and mixtures thereof.

The oral care composition of the present invention may include a preservative. Although benzyl alcohol may be considered a preservative, in certain embodiments, the composition further comprises other preservatives. Suitable preservatives include, but are not limited to, sodium benzoate, potassium sorbate, methylisothiazolinone, paraben preservatives, for example methyl p-hydroxybenzoate, propyl p-hydroxybenzoate, and mixtures thereof.

The oral care composition of the invention may include an antioxidant. Any orally acceptable antioxidant may be used, including, but not limited to, butylated hydroxyanisole (BHA), butylated hydroxytoluene (BHT), vitamin A, carotenoids, vitamin E, flavonoids, polyphenols, ascorbic acid, herbal antioxidants, chlorophyll, melatonin, or the like, or combinations and mixtures thereof.

The oral care composition of the invention may include one or more pigments, such as whitening pigments. In some embodiments, the whitening pigments include particles ranging in size from about 0.1 µm to about 10 µm with a refractive index greater than about 1.2. Suitable whitening agents include, without limitation, titanium dioxide particles, zinc oxide particles, aluminum oxide particles, tin oxide particles, calcium oxide particles, magnesium oxide particles, barium oxide particles, silica particles, zirconium silicate particles, mica particles, talc particles, tetracalcium phosphate particles, amorphous calcium phosphate particles, alpha-tricalcium phosphate particles, beta-tricalcium phosphate particles, hydroxyapatite particles, calcium carbonate particles, zinc phosphate particles, silicon dioxide particles, zirconium silicate particles, or the like, or mixtures and combinations thereof. The whitening pigment, such as titanium dioxide particles, may be present in an amount that is sufficient to whiten the teeth.

As noted above, the oral care compositions may comprise a first component, such as the oral care whitening composition disclosed herein, and a second component, such as the pH adjuster compositions disclosed herein. The oral care composition may form at least a portion of or be used in one or more oral care products. Illustrative oral care products may include, but are not limited to, a toothpaste (dentifrice), a prophylactic paste, a tooth polish, a tooth gel (e.g., whitening gel), a chewing gum, a lozenge, a paint-on gel, varnish, veneer, and tube, syringe or pump, or dental tray comprising a gel as described herein, or a gel coated on an application support such as dental floss, interdental brush, dental pad, or a toothbrush (e.g., a manual, electric, sound, a combination thereof or ultrasound. In some embodiments, the oral care composition is a toothpaste gel, or serum. The oral care composition and/or product may be in the form of a gel. For example, the oral care composition and/or product may comprise a first component that forms a first gel phase and a second component that forms a second gel phase.

The oral care compositions may be prepared by first producing an oral care whitening composition component and a pH adjuster composition component. In some embodiments, the oral care compositions are formulated to have a mass ratio the first component (e.g., an oral care whitening composition component) to the second component (e.g., the pH adjuster composition component) of from about 1:10 to about 10:1. In some instances, the mass ratio of the first component (e.g., an oral care whitening composition component) to the second component (e.g., the pH adjuster composition component) is from about 1:10 to about 10: 1, about 1:7 to about 10:1, about 1:5 to about 10:1, about 1:3 to about 10:1, about 1:2 to about 10:1; about 1:10 to about 7:1, about 1:7 to about 7:1, about 1:5 to about 7:1, about 1:3 to about 7:1, about 1:2 to about 7:1; about 1:10 to about 5:1, about 1:7 to about 5:1, about 1:5 to about 5:1, about 1:3 to about 5:1, about 1:2 to about 5:1; about 1:10 to about 3:1, about 1:7 to about 3:1, about 1:5 to about 3:1, about 1:3 to about 10: 1, about 1:2 to about 3:1; about 1:7 to about 7:1, about 1:6 to about 6:1, about 1:5 to about 5:1, about 4:1 to about 1:4, about 1:3 to about 3:1, about 2:1 to about 1:2, or about 1:1

The whitening composition may comprise a low level (e.g., less than about 1%, less than about 0.8%, less than about 0.5%,) of a peroxide. The oral care compositions provide for beneficial whitening efficacy and have compositional stability. In certain embodiments, the whitening composition comprises: a peroxide source present in an amount to provide from about 0.1% to about 1% of a peroxide source by weight of the composition; a polyacrylic acid polymer present in an amount from about 1% to about 5% by weight of the composition; a poloxamer present in an amount from about 0.2% to about 1% by weight of the composition; benzyl alcohol present in an amount from about 0.2% to about 1% by weight of the composition; and an orally acceptable carrier present in an amount from about 80% to about 97% by weight of the composition; wherein the pH of the composition is from about 5 to about 6. In certain embodiments, the whitening composition comprises a peroxide source present in an amount to provide from about 0.1% to about 1% of hydrogen peroxide by weight of the composition; a synthetic polyacrylic acid polymer present in an amount from about 1% to about 5% by weight of the composition; a poloxamer present in an amount from about 0.2% to about 1% by weight of the composition; benzyl alcohol present in an amount from about 0.2% to about 1% by weight of the composition; sodium hydroxide present in an amount from about 0.3% to about 0.45% by weight of the composition; and an orally acceptable carrier present in an amount from about 40% to about 97% by weight of the composition; wherein the pH of the composition is from about 5 to about 6. The ingredients and/or components of the whitening composition may be selected from any of those described herein with reference to the oral care composition. For example, the whitening composition may include any of the peroxide sources, polyacrylic acids, poloxamers, bases, etc. described herein with reference to the oral care composition.

The pH of the oral care whitening composition (first component) may vary. However, having a pH too low or too high may negatively affect the efficacy and/or stability of the composition. In certain embodiments, the pH of the whitening composition is from about 5 to about 6. In certain embodiments, the pH of the whitening composition is from about 5.1 to about 5.8, from about 5.2 to about 5.7, or from about 5.2 to about 5.5.

The pH adjuster composition (second component) may comprise a thickening agent; a carbonate; a bicarbonate; and an orally acceptable carrier present in an amount from about 80% to about 97% by weight of the composition; wherein the pH of the second composition is from about 10 to about 11, wherein the mass ratio of carbonate to bicarbonate is from about 12:1 to about 4:1; and wherein the second composition has a viscosity from about 200,000 to about 310,000 cp.

The pH adjuster composition may comprise a thickener; a carbonate; a bicarbonate; and an orally acceptable carrier present in an amount from about 80% to about 97% by weight of the composition; wherein the pH of the second composition is from about 10 to about 11, wherein the mass ratio of carbonate to bicarbonate is from about 12:1 to about 4:1; and wherein the second composition has a viscosity from about 200,000 to about 310,000 cp. The ingredients and/or components of the pH composition may be selected from any of those described herein with reference to the oral care composition. For example, the pH composition may include any of the thickeners, orally acceptable carriers, bases, etc. described herein with reference to the oral care composition.

In certain embodiments, the pH adjuster composition has a viscosity from about 210,000 to about 290,000 cp. In certain embodiments, the mass ratio of carbonate to bicarbonate is from about 10:1 to about 5:1, from about 10:1 to about 7:1, from about 9.5:1 to about 7.5:1, or from about 8:1 to about 9:1. In certain embodiments, the carbonate is sodium carbonate. In certain embodiments, the bicarbonate is sodium bicarbonate.

The thickening agent of the pH adjuster composition may vary. In certain embodiments, the thickening agent of the second composition is selected from carboxyvinyl polymers, carrageenan, hydroxyethyl cellulose, laponite, water soluble salts of cellulose ethers such as sodium carboxymethylcellulose and sodium carboxymethyl hydroxyethyl cellulose, natural gums such as gum karaya, xanthan gum, gum arabic, and gum tragacanth and combinations thereof. In certain embodiments, the thickening agent of the second composition is carboxymethylcellulose. In certain embodiments, the thickening agent of the second composition is present in an amount from about 1% to about 8%, from about 2.5% to about 6%, or from about 4% to about 6% by weight of the second composition.

The pH adjuster composition may further comprise one or more of the additional ingredients, including those discussed above. For example, in at least one embodiment, the pH adjuster includes a sweetener. The sweetener may comprise the same, similar, or different sweeteners and amounts as in the oral care composition. In certain embodiments, the second composition comprises a sweetener present in an amount from about 0.05% to about 1%, from about 0.1% to about 0.8%, or from about 0.4% to about 0.6%, by weight of the second composition.

The pH of the pH adjuster composition may vary. However, having a pH too low or too high may negatively affect the efficacy and/or stability of the oral care composition. In certain embodiments, the pH of the pH adjuster composition is from about 9.5 to about 11.5. In certain embodiments, the pH of the pH adjuster composition is from about 9.8 to about 11.3, from about 10 to about 11, or from about 10.2 to about 10.8.

In at least one implementation, the viscosity or target viscosity of the pH adjuster composition may be greater than or equal to about 210,000 cP and less than or equal to about 320,000 cP. For example, the viscosity of the pH adjuster composition may be about 210,000 cP, about 220,000 cP, about 250,000 cP, about 260,000 cP, about 270,000 cP, about 290,000 cP, about 300,000 cP, about 310,000 cP, or about 220,000 cP to about 310,000 cP.

The pH adjuster may be applied to a tooth surface before, during, or after the whitening composition. As such, in an aspect of the invention, a method of whitening teeth comprises applying a pH adjuster composition to the surface of the teeth after a whitening composition as described herein has been applied. In certain aspects, the oral care composition may be used to form an oral care product composition by mixing the whitening composition with the pH adjuster composition as described herein. Alternatively, being that the oral care product composition has superior compositional stability, the oral care product may be stored until use. Therefore, an aspect of the invention is a method of whitening teeth as described above, wherein prior to applying to the surface of the teeth, a first amount of the whitening composition and about an equal amount of the pH adjuster composition are mixed together.

In other aspects, provided is a method of forming an oral care product composition by mixing a whitening composition as described herein with about an equal amount of a pH adjuster composition as described herein. Without being bound by theory, it is believed that by separating the compositions and mixing them just prior to application on the teeth, the pH of the low peroxide aqueous gel increases, generally to a value of about 9. In such an alkaline solution level, hydrogen peroxide may dissociate to hydrogen cation (H+) and perhydroxyl anion (HO2-). Perhydroxyl anion is an important active species which may react with the stain to produce a whitening effect. When the compositions are mixed together to form a single composition, the peroxide may degrade within 24 hours. Thus, by maintaining the compositions separately and mixing prior to intended use, compositional stability and efficacy is maintained during storage conditions.

In further aspects, the invention is a kit comprising an oral care whitening composition (first component) as described herein and a pH adjuster composition (second component). The kit is useful for storage of the compositions. Upon intended use, the compositions may be mixed to produce an oral care product.

Various non-limiting embodiments of the disclosure are provided below. In one embodiment, an oral care composition (Composition 1.0) comprising a peroxide source present in an amount to provide from about 0.1% to about 1% of peroxide by weight of the composition, a synthetic polyacrylic acid polymer present in an amount from about 1% to about 5% by weight of the composition; a poloxamer present in an amount from about 0.2% to about 1% by weight of the composition; benzyl alcohol present in an amount from about 0.2% to about 1% by weight of the composition; and an orally acceptable carrier present in an amount from about 80% to about 97% by weight of the composition; wherein the pH of the composition is from about 5 to about 6.

In certain embodiments, the invention includes a Composition 1.1, wherein the composition comprises a peroxide source present in an amount to provide from about 0.1% to about 1% of hydrogen peroxide by weight of the composition; a synthetic polyacrylic acid polymer present in an amount from about 1% to about 5% by weight of the composition; a poloxamer present in an amount from about 0.2% to about 1% by weight of the composition; benzyl alcohol present in an amount from about 0.2% to about 1% by weight of the composition; and an orally acceptable carrier present in an amount from about 80% to about 97% by weight of the composition; wherein the pH of the composition is from about 5 to about 6. In certain embodiments, the invention is Composition 1.2 or Composition 1.1, wherein the peroxide source is selected from hydrogen peroxide, a peroxide whitening complex, and combinations thereof. Composition 1.3, one of Compositions 1.1 or 1.2, wherein the peroxide source is present in an amount to provide about 0.1% to about 0.5% by weight of the composition. In Composition 1.4, any Composition 1.1 to 1.3, wherein the synthetic polyacrylic acid polymer is a carbomer. In Composition 1.5, any of Compositions 1.1 to 1.4, wherein the synthetic polyacrylic acid polymer is present in an amount from about 1% to about 3.5% by weight of the composition. In Composition 1.6, any of Compositions 1.1 to 1.5, wherein the poloxamer is a non-ionic surfactant composed of blocks of polyethylene glycol (PEG) and polypropylene glycol (PPG). In Composition 1.7, any of Compositions 1.1 to 1.6, wherein the poloxamer is present in an amount from about 0.3% to 0.8% by weight of the composition. In Composition 1.8, any of Compositions 1.1 to 1.7, wherein the benzyl alcohol is present in an amount from about 0.2 to about 1 % by weight of the composition. In Composition 1.9, any Composition 1.1 to 1.8, wherein the orally acceptable carrier is selected from glycerin, water, polymers and/or copolymers of polyethylene glycol, of ethylene oxide propylene oxide, and of silicone, and combinations thereof. In Composition 1.10, the Composition 1.9, wherein the orally acceptable carrier is present in an amount from about 90% to about 97% by weight of the composition. In Composition 1.11, any of Compositions 1.1 to 1.10, wherein the composition further comprises sodium hydroxide. In Composition 1.12, the Composition 1.11, wherein the sodium hydroxide is present in an amount from about 0.3% to about 0.45% by weight of the composition. In Composition 1.13, any of Compositions 1.1 to 1.11, wherein the composition further comprises a sweetener, present in an amount from about 0.05% to about 0.4%, from about 0.08% to about 0.3%, or from about 0.1% to about 0.3%, by weight of the composition. In Composition 1.14, any of Compositions 1.1 to 1.13 wherein the pH of the composition is from about 5.2 to about 5.5. In Composition 1.15, any of Compositions 1.1 to 1.14, wherein the composition further comprises peppermint flavor. In Composition 1.16, Composition 1.15, wherein the peppermint flavor is present in an amount from about 0.05% to about 0.4%, from about 0.08% to about 0.3%, or from about 0.1% to about 0.3%, by weight of the composition. An oral care product 1.17 comprising: mixing a first amount of any of Compositions 1.1 to 1.16 with about an equal amount of a second composition (Composition 2.0) comprising a thickening agent; a carbonate; a bicarbonate; and an orally acceptable carrier present in an amount from about 80% to about 97% by weight of the composition; wherein the pH of the second composition is from about 10 to about 11, wherein the mass ratio of carbonate to bicarbonate is from about 12:1 to about 4:1; and wherein the second composition has a viscosity from about 200,000 to about 310,000 cp. In oral care product 1.18, oral care product 1.17, wherein the pH is from about 9 to about 10. In oral care product 1.19, one of oral care products 1.17 or 1.18, wherein the second composition (Composition 2.0) has a viscosity from about 210,000 to about 290,000 cp. In oral care product 1.20, any of oral care products 1.17 to 1.19, wherein the mass ratio of carbonate to bicarbonate is from about 10:1 to about 7:1. In oral care product 1.21, any of oral care products 1.17 to 1.20, wherein the thickening agent of the second composition (Composition 2.0) is selected from carboxyvinyl polymers, carrageenan, hydroxyethyl cellulose, laponite, water soluble salts of cellulose ethers such as sodium carboxymethylcellulose and sodium carboxymethyl hydroxyethyl cellulose, natural gums such as gum karaya, xanthan gum, gum arabic, and gum tragacanth and combinations thereof. In oral care product 1.22, oral care product 1.21, wherein the thickening agent of the second composition is present in an amount from about 1% to about 8% by weight of the composition. In oral care product 1.23, any of oral care products 1.17 to 1.22, wherein the second composition further comprises a sweetener, present in an amount from about 0.05% to about 1%, from about 0.1% to about 0.8%, or from about 0.4% to about 0.6%, by weight of the composition.

In certain embodiments, the invention includes a method (Method 1.24) of whitening teeth, comprising applying any of Compositions 1.1 to 1.23 to the surface of the teeth. In certain embodiments, the invention is Method 1.40, which includes Method 1.24, wherein prior to applying to the surface of the teeth, a first amount of the oral care composition (any Composition 1.1 to 1.16) and about an equal amount of a second composition (Composition 2.0) are mixed together. In method 1.41, the method of 1.40, wherein the whitening composition is mixed with the second composition in a weight ratio of about 1:7 to about 7:1. In method 1.42, the method of 1.40, wherein the pH of the mixed composition is from about 9 to about 10. In the method of 1.43, any of method 1.40 to 1.42, wherein the second composition has a viscosity from about 210,000 to about 290,000 cP. In method 1.44, any one of methods 1.40 to 1.42, wherein the mass ratio of carbonate to bicarbonate is from about 10:1 to about 7:1. In method 1.45, any one of method 1.40 to 1.44, wherein the thickening agent of the second composition is selected from carboxyvinyl polymers, carrageenan, hydroxyethyl cellulose, laponite, water soluble salts of cellulose ethers such as sodium carboxymethylcellulose and sodium carboxymethyl hydroxyethyl cellulose, natural gums such as gum karaya, xanthan gum, gum arabic, and gum tragacanth and combinations thereof. In method 1.46, the method of 1.44, wherein the thickening agent of the second composition is present in an amount from about 1% to about 8% by weight of the composition. I method 1.47, any one of method 1.40 to 1.45, wherein the second composition further comprises a sweetener, present in an amount from about 0.05% to about 1%, from about 0.1% to about 0.8%, or from about 0.4% to about 0.6%, by weight of the composition.

In certain embodiments, provided is an oral care product 1.48 comprising a first component comprising: a peroxide source in an amount of from about 0.01 to about 2 wt.%; benzyl alcohol present in an amount from about 0.2 to about 3 wt.%; optionally, a second base; a second component comprising: a first base in an amount up to about 5 wt.%; wherein the oral care product after mixing of the first component and the second component has a pH of about 8 to about 12, and all weight percentages are based on the total weight of the oral care product. In oral care product 1.49. the oral are product of 1.48, wherein the first base comprises sodium hydroxide, a carbonate, a bicarbonate, or a mixture thereof. In oral care product 1.50, the oral care product of 1.48 or 1.49, wherein the first base comprises a carbonate and a bicarbonate. In oral care product 1.51, the oral care product of 1.50 or 1.51, wherein the oral care product has a mass ratio of the carbonate to the bicarbonate that is from about 12:1 to about 4:1. In oral care product 1.52, any of the oral products of 1.48 to 1.51 further comprising a thickener in an amount from about 1 to about 10 wt.%. In oral care product 1.53, the oral care product of 1.52, wherein the thickener is selected from a polyacrylic polymer, a poloxamer, a polysaccharide, a carboxymethyl cellulose, and a combination of two or more thereof. In oral care product 1.54, the oral care product of 1.52, wherein the thickener comprises a poloxamer. In oral care product 1.55, the oral care product of 1.52, wherein the thickener comprises two or more of a poloxamer, a carboxymethyl cellulose, and a polyacrylic polymer. In oral care product 1.56, any of oral care products of 1.48 to 1.55, wherein the oral care product is a two-phase composition. In oral care product 1.57, the oral care product of 1.56, wherein the first component forms a first phase and the second component forms a second phase of the two-phase composition, the first phase and the second phase being in contact at a single interphase. In oral care product 1.58, any of oral care products 1.48 to 1.55, wherein the first component and the second component are physically separated. In oral care product 1.59, the oral care product of 1.48, wherein the first component further comprises a mint flavor in an amount up to about 3 wt.%.

In certain non-limiting embodiments, the oral care composition may comprise a formulation as specified in Table 1a below. In certain non-limiting embodiments, the oral care product may comprise a mixture of about 1:3 to about 3:1, or about 2:1 to about 1:2, or about 1:1, by weight, of the formulations specified in Tables 1a and 1b, respectively.

TABLE 1

Exemplary, Non-limiting Oral Care Composition

| Component | Wt.% |
|---|---|
| Glycerin | 30 |
| Water | Q.S. to 100 |
| Hydrogen Peroxide Solution | 0.01-1 |
| CMC | 2.6 |
| Carbomer and Poloxamer | 1.3 |
| Sodium Saccharin | 0.4 |
| Peppermint Flavor | 0.1 |
| Benzyl Alcohol | 0.3 |
| Sodium Hydroxide | 0.2 |
| Sodium Carbonate and Sodium Bicarbonate | 1.6 |

TABLE 1a

Exemplary, Non-limiting Whitening Composition

| Component | wt. % |
|---|---|
| Glycerin | 25-97 |
| Water | 60-97 |
| Peroxide Source | 0.1-1 |
| Synthetic Polyacrylic Acid Polymer | 1-5 |
| Sweetener | 0-1 |
| Poloxamer | 0.2-1 |
| Benzyl Alcohol | 0.2-1 |
| Sodium Hydroxide | 0-0.5 |
| Peppermint Flavor | 0-0.4 |
| pH | 5-6 |
| Viscosity (cP) | 200,000-310,000 |

TABLE 1b

Exemplary Non-limiting Buffer Gel Composition

| Component | wt. % |
|---|---|
| Thickening Agent | 1-8 |
| Carbonate | 1-6 |
| Bicarbonate | 0.1-1 |
| Sweetener | 0-1 |
| Glycerin | 25-97 |
| Water | 55-97 |
| Carbonate: Bicarbonate | 12:1-4:1 |
| pH | 10-11 |
| Viscosity (cP) | 220,000-310,000 |

### EXAMPLES

The examples and other implementations described herein are exemplary and not intended to be limiting in describing the full scope of compositions and methods of this disclosure. Equivalent changes, modifications and variations of specific implementations, materials, compositions, and methods may be made within the scope of the present disclosure, with substantially similar results.

### Example 1

Whitening toothpaste compositions having formulations as indicated in Table 2 were prepared. All compositions were in the form of a clear gel and contained a final hydrogen peroxide concentration of about 0.2 wt.%, based on the total weight of the whitening composition.

TABLE 2

Whitening Compositions

| Component | Comp. Example 1 (wt. %) | Comp. Example 2 (wt. %) | Comp. Example 3 (wt. %) | Test Example 1 (wt. %) | Test Example 2 (wt. %) |
|---|---|---|---|---|---|
| Glycerin | 30 | 30 | 30 | 30 | 30 |
| Water | 65.07 | 65.23 | 65.33 | 65.43 | 65.23 |
| Hydrogen Peroxide (35% Solution) | 0.57 | 0.57 | 0.57 | 0.57 | 0.57 |
| Carbomer | 2 | 2 | 2 | 2 | 2 |
| Sodium Saccharin | 0.2 | 0.2 | 0.2 | 0.2 | 0.2 |
| Poloxamer | 0.5 | 0.5 | 0.5 | 0.5 | 0.5 |
| Benzyl Alcohol | 0.5 | 0.5 | 0.5 | 0.5 | 0.5 |
| Sodium Hydroxide (50% Solution) | 1.16 | 1 | 0.9 | 0.8 | 0.8 |
| Peppermint Flavor | | | | | 0.20 |
| Total Components | 100 | 100 | 100 | 100 | 100 |
| Viscosity (cP) | 315,465 | 241,551 | 284,004 | 228,874-279,823 | 302,160 |
| pH | 6.7 | 6.04 | 5.69 | 5.36-5.59 | 5.4 |

As shown within Table 2, surprisingly and unexpectedly, the amount of sodium hydroxide present affected both the pH and viscosity of the final oral care composition. While comparative Examples 1 to 3 exhibited pH values of about 5.69 or higher, utilization of lower amounts of sodium hydroxide provided for lower pH values.

### Example 2

To characterize stability of the compositions, stress tests were performed at various temperatures for up to 4 weeks and the pH, viscosity, and active oxygen monitored. Test Examples 1 and 2 displayed attributes as shown in Tables 3 and 4, respectively.

TABLE 3

Stress test results for Test Example 1.

| Temperature | Time | pH of 10% Solution | Viscosity (cP) | Active Oxygen (%) |
|---|---|---|---|---|
| Room Temperature | Initial | 6.40 | 228,874 | 0.18 |
| 40 °C | 1 week | 6.03 | 262,665 | 0.19 |
| | 2 weeks | 6.16 | 172,754 | 0.16 |
| | 4 weeks | 6.09 | 126,656 | 0.15 |
| 49 °C | 1 week | 6.13 | 209,535 | 0.17 |
| | 2 weeks | 6.33 | 160,953 | 0.14 |
| | 4 weeks | 5.50 | 6,671 | 0.00 |
| 60 °C | 1 week | 5.46 | 36,700 | N/A |
| | 2 weeks | 6.03 | 27,150 | N/A |
| | 4 weeks | 5.52 | 3,707 | N/A |

TABLE 4

Stress test results for Test Example 2.

| Temperature | Time | pH of 10% Solution | Viscosity (cP) | Active Oxygen (%) |
|---|---|---|---|---|
| Room Temperature | Initial | 6.2 | 302,160 | 0.2 |
| 40 °C | 1 week | 6.33 | 300,586 | 0.2 |
| | 2 weeks | 6.83 | 336,736 | 0.19 |
| | 4 weeks | 6.38 | 350,221 | 0.18 |
| 49 °C | 1 week | 6.72 | 259,560 | 0.21 |
| | 2 weeks | 6.59 | 233,376 | 0.19 |
| | 4 weeks | 6.39 | 217,534 | 0.17 |
| 60 °C | 1 week | 6.39 | 309,594 | N/A |
| | 2 weeks | 6.28 | 250,645 | N/A |
| | 4 weeks | 6.14 | 65,497 | N/A |

As shown in Table 3, Test Example 1 had a drop in viscosity and active oxygen level at temperatures as low as 40° C. At 49° C., the viscosity and active oxygen level saw dramatic loss in viscosity and active oxygen level. In contrast, Test Example 2, which contained peppermint flavor, displayed stable pH, viscosity, and active oxygen levels under similar conditions.

### Example 3

Preparation of a buffer gel (also referred to as a second composition) and stability studies. To enhance the stability of the whitening toothpaste composition, a buffer gel was prepared as described within Table 5.

TABLE 5

Gel buffer composition.

| Component | wt. % |
|---|---|
| Glycerin | 30 |
| Sodium Carboxymethylcellulose (CMC) | 5 |
| Sodium Carbonate | 2.781 |
| Sodium Bicarbonate | 0.315 |
| Sodium Saccharin | 0.5 |
| Water | q.s. |
| Total | 100 |

The resulting gel buffer composition (as described in Table 5) had a viscosity of about 250,000 cP and pH value of about 10.59. The gel buffer was mixed with the composition of Test Example 2 at about a 1:1 weight ratio to produce Test Example 3. Test Example 3 had a pH value of about 9 to 10. Test Example 3 was tested for stability at various temperatures in either a glass vial or a polyethylene container. The stability results are shown in Table 6.

TABLE 6

Stability evaluation of Test Example 3

| Temperature | Time | pH of 10% Solution | | Viscosity (cP) | |
|---|---|---|---|---|---|
| | | Glass Vial | Polyethylene container | Glass Vial | Polyethylene container |
| -10° C. | 4 weeks | 10.70 | 10.74 | 143,859 | 139,396 |
| | 8 weeks | 10.31 | 10.74 | 170,110 | 144,565 |
| | 13 weeks | 10.05 | 10.54 | 162,369 | 191,257 |
| 30° C. | 4 weeks | 10.75 | 10.65 | 172,403 | 160,853 |
| | 8 weeks | 10.74 | 10.65 | 196,741 | 162,234 |
| | 13 weeks | 10.50 | 10.74 | 241,345 | 183,857 |
| 40° C. | 4 weeks | 10.74 | 10.60 | 194,999 | 172,513 |
| | 8 weeks | 10.65 | 10.65 | 291,798 | 177,002 |
| | 13 weeks | 10.68 | 10.55 | 357,353 | 195,693 |
| 49° C. | 4 weeks | 10.72 | 10.65 | 207,721 | 176,060 |
| | 8 weeks | 10.58 | 10.70 | 320,327 | 172,450 |
| | 13 weeks | 10.34 | 10.77 | 368,998 | 205,577 |

As shown in Table 6, Test Example 3 shows stable pH and viscosity within both glass and polyethylene vessels at temperatures up to and including 49° C.

### Example 4

Testing of effect of carbonate/bicarbonate buffer on tooth whitening efficacy. Test Example 3 was tested against Comparative Example 4 for effects on tooth whitening. Comparative Example 4 was prepared by mixing a placebo buffer mixture (comprising 30 wt. % glycerin, 5 wt. % CMC, 0.50 wt. % sodium saccharin, water, pH value about 5.56, viscosity about 260,000 cP) with the composition of Test Example 2 at about a 1:1 weight ratio, which resulted in a final pH of about 5.2. Test Example 3 had a final pH of about 9 to about 10. The testing was performed throughout 5 days with 1 treatment per day. Results of the tooth whitening experiment are reported in Table 7.

TABLE 7

Evaluation of tooth whitening effects (as determined by delta W)

| | Treatment 1 | Treatment 2 | Treatment 3 | Treatment 4 | Treatment 5 |
|---|---|---|---|---|---|
| Comparative Example 4 | -0.6 | -1.1 | -1.5 | -2.6 | -2.7 |
| Test Example 3 | -1.1 | -3 | -4.2 | -6.4 | -7.2 |

As shown in Table 7, Test Example 3 showed significantly greater whitening performance than Comparative Example 4. Without being limited to any particular theory, it is believed that the unexpected increase in whitening performance by Test Example 3 was at least partially due to the combination of the pH of the final toothpaste composition, benzyl alcohol in the whitening composition, and the gel buffer composition.

### Example 5

Testing of effect of exemplary final toothpaste composition on tooth whitening efficacy. Test Example 3 was tested against Comparative Example 5 for effects on tooth whitening. Comparative Example 5 was prepared by mixing a composition as described in Comparative Example 1, but lacking benzyl alcohol, with the gel buffer composition at about a 1:1 weight ratio. The testing was performed throughout 5 days with 1 treatment per day. Average results (n = 6) of the tooth whitening experiment are reported in Table 8.

TABLE 8

Evaluation of tooth whitening effects (as determined by delta W)

| | Treatment 1 | Treatment 2 | Treatment 3 | Treatment 4 | Treatment 5 |
|---|---|---|---|---|---|
| Comparative Example 5 | -0.6 | -1.8 | -3.1 | -4.5 | -6.6 |
| Test Example 3 | -1.1 | -3 | -4.2 | -6.4 | -7.2 |

As shown in Table 8, Test Example 3 showed significantly greater whitening performance than Comparative Example 5.

While the present invention has been described with reference to several embodiments, which embodiments have been set forth in considerable detail for the purposes of making a complete disclosure of the invention, such embodiments are merely exemplary and are not intended to be limiting or represent an exhaustive enumeration of all aspects of the invention. The scope of the invention is to be determined from the claims appended hereto. Further, it will be apparent to those of skill in the art that numerous changes may be made in such details without departing from the spirit and the principles of the invention.

1. An oral care composition comprising:
a peroxide source present in an amount to provide from about 0.1 to about 1 wt.% of peroxide, based on the total weight of the oral care composition;
a synthetic polyacrylic acid polymer present in an amount from about 1 to about 5 wt.%, based on the total weight of the oral care composition;
a poloxamer present in an amount from about 0.2 to about 1 wt.%, based on the total weight of the oral care composition;
benzyl alcohol present in an amount from about 0.2 to about 1 wt.%, based on the total weight of the oral care composition; and
an orally acceptable carrier present in an amount from about 80 to about 97 wt.%, based on the total weight of the oral care composition,
wherein the pH of the oral care composition is from about 5 to about 6.

2. The oral care composition of claim 1, wherein the peroxide source is selected from hydrogen peroxide, a peroxide whitening complex, and combinations thereof.

3. (canceled)

4. The oral care composition of claim 1, wherein the synthetic polyacrylic acid polymer is a carbomer.

5. (canceled)

6. The oral care composition of claim 1, wherein the poloxamer is a non-ionic surfactant composed of blocks of polyethylene glycol (PEG) and polypropylene glycol (PPG).

7-16. (canceled)

17. An oral care product comprising mixing a first amount of a first oral care composition according to claim 1 with about an equal amount of a second composition comprising:
a thickening agent;
a carbonate;
a bicarbonate; and
an orally acceptable carrier present in an amount from about 80 to about 97 wt.% based on the total weight of the second oral care composition;
wherein the pH of the second composition is from about 10.0 to about 11.0,
wherein the mass ratio of carbonate to bicarbonate is from about 12:1 to about 4:1; and
wherein the second composition has a viscosity from about 200,000 to about 310,000 cP.

18-19. (canceled)

20. The oral care product of claim 17, wherein the mass ratio of carbonate to bicarbonate is from about 10:1 to about 7:1.

21. The oral care product of claim 17, wherein the thickening agent of the second composition is selected from carboxyvinyl polymers, carrageenan, hydroxyethyl cellulose, laponite, sodium carboxymethylcellulose, sodium carboxymethyl hydroxyethyl cellulose, gum karaya, xanthan gum, gum arabic, gum tragacanth, and combinations thereof.

22-23. (canceled)

24. A method of whitening teeth, the method comprising: applying to the surface of the teeth an oral care whitening composition comprising:
a peroxide source present in an amount to provide from about 0.1 to about 1 wt.% of peroxide, based on the total weight of the oral care whitening composition;
a synthetic polyacrylic acid polymer present in an amount from about 1 to about 5 wt.%, based on the total weight of the oral care whitening composition;
a poloxamer present in an amount from about 0.2 to about 1 wt.%, based on the total weight of the oral care whitening composition;
benzyl alcohol present in an amount from about 0.2 to about 1 wt.%, based on the total weight of the oral care whitening composition; and
an orally acceptable carrier present in an amount from about 80 to about 97 wt.%, based on the total weight of the oral care whitening composition,
wherein the pH of the oral care whitening composition is from about 5 to about 6.

25-47. (canceled)

48. An oral care product comprising:
a first component comprising:
a peroxide source in an amount of from about 0.01 to about 2 wt.%;
benzyl alcohol present in an amount from about 0.2 to about 3 wt.%;
optionally, a second base; and
a second component comprising:
a first base in an amount up to about 5 wt.%,
wherein the oral care product after mixing of the first component and the second component has a pH of about 8 to about 12, and all weight percentages are based on the total weight of the oral care product.

49. The oral care product of claim 48, wherein the first base comprises sodium hydroxide, a carbonate, a bicarbonate, or a mixture thereof.

50. The oral care product of claim 48 , wherein the first base comprises a carbonate and a bicarbonate.

51. The oral care product of claim 50, wherein the oral care product has a mass ratio of the carbonate to the bicarbonate that is from about 12:1 to about 4:1.

52. The oral care product of claim 48 further comprising a thickener in an amount from about 1 to about 10 wt.%.

53. The oral care product of claim 52, wherein the thickener is selected from a polyacrylic polymer, a poloxamer, a polysaccharide, a carboxymethyl cellulose, and a combination of two or more thereof.

54. The oral care product of claim 52, wherein the thickener comprises a poloxamer.

55. The oral care product of claim 52, wherein the thickener comprises two or more of a poloxamer, a carboxymethyl cellulose, and a polyacrylic polymer.

56. The oral care product of claim 48, wherein the oral care product is a two-phase composition.

57. The oral care product of claim 56, wherein the first component forms a first phase and the second component forms a second phase of the two-phase composition, the first phase and the second phase being in contact at a single interphase.

58. The oral care product of claim 48, wherein the first component and the second component are physically separated.

59. The oral care product of claim 48, wherein the first component further comprises a mint flavor in an amount up to about 3 wt.%.
